(19) United States
(12) Patent Application Publication
(10) Pub. No.: US 2021/0161696 A1
(43) Pub. Date: Jun. 3, 2021

(54) FINGER GLOVE LINER AND STRENGTHENING FINGER GLOVE WITH LINER

(71) Applicant: Bioservo Technologies Aktiebolag, Kista (SE)

(72) Inventors: Martin Oskar Gustaf EWALDSSON, Sigtuna (SE); Lars FRODE, Stockholm (SE); Carl Joakim GABRIELSSON, Täby (SE); Emma Helena LINDER, Uppsala (SE)

(21) Appl. No.: 16/304,155

(22) PCT Filed: May 29, 2017
(86) PCT No.: PCT/EP2017/062932

§ 371 Date: Nov. 22, 2018

(30) Foreign Application Priority Data
May 30, 2016 (SE) 1650751-9

(51) Int. Cl. A61F 5/01; A41D 19/015

(52) U.S. Cl. CPC A61F 5/013; A61F 2005/0155; A41D 19/01547

(57) ABSTRACT

A finger glove liner (2) comprising at least one finger portion (1), for arrangement inside an outer finger glove, making up a grip strengthening finger glove (3) is provided. The finger glove liner (2) is made of a stretchable material (4) in order to suit more than one size of hands, wherein at least one patch (5) of material, which has attached thereto at least an artificial tendon (9), is arranged at the outside of the finger glove liner (2).

### TECHNICAL FIELD

The present invention concerns finger glove liner, for arrangement inside an outer finger glove, making up a grip strengthening finger glove having at least one finger portion for enhancing the grip for at least one corresponding finger of a wearer by means of at least one artificial tendon arranged along at least one finger portion of the grip strengthening support device.

### BACKGROUND

There are previously known grip strengthening support devices, for example shown in WO 2008/027002. In this document a finger glove for grip strengthening is shown, having at least one glove finger with at least one artificial tendon on the inside of the glove finger on both sides of the glove finger and the artificial tendon runs in a duct attached on the inside of the glove finger. When a pulling force is applied to the artificial tendon the glove finger will bend towards the palm of the glove, i.e. the ventral side of the glove.

It is cumbersome attaching and arranging the artificial tendons and guiding means therefor on the inside of a finger glove. Additionally, sensors and wires need also to be attached. Another way of arranging and attaching artificial tendons, sensors and wires is to arrange and attach them at a liner which thereafter is covered by a finger glove. It is still a rather work extensive handling much made by hand and in many different sizes, thus causing an expensive product.

### SUMMARY OF THE INVENTION

It is an aim of the present invention to provide a grip strengthening finger glove which may be produced in an easier way and partially in less different sizes.

According to a first aspect of the invention a finger glove liner comprising at least one finger portion, for arrangement inside an outer finger glove, making up a grip strengthening finger glove is provided. The finger glove liner is made of a stretchable material in order to suit more than one size of hands. At least one patch of material is arranged at the outside of the finger glove liner. At least one artificial tendon is attached to the at least one patch. This makes it possible to produce less sizes of the inventive finger glove liner and still provide a suitable fit and the same kind of effective gripping when the at least one artificial tendon is pulled. It is not possible to attach the at least one artificial tendon directly to the stretchable liner material as this would stretch instead of taking up the forces provided by the at least one artificial tendon.

According to one embodiment the at least one patch of material is made of a flexible and non-stretchable material in a direction crosswise the length direction of the finger portion. Thus the at least one patch can distribute the force from the at least one artificial tendon attached to said patch. The force from the artificial tendons will thus be transferred via the patches to a finger of a wearer thus creating a bending force on the finger, which will enhance the gripping of the wearer.

According to one embodiment the patches are arranged freely from each other in order to be able to transfer with the expansion of the stretchable liner in use. In this way the patches will be in the right position even if a bigger hand is put into the stretchable finger glove liner. According to one embodiment the at least one patch is arranged on a dorsal side of the finger glove liner. Preferably the at least one patch reaches over each of the sides of a finger of the finger glove liner with a side portion on each side. Then it is possible to arrange the at least one artificial tendon at a side portion, preferably along the edge of the side portion of the patch.

According to one embodiment at least one sensor is arranged at at least one patch. Thus the grip strengthening finger glove may sense when a wearer are going to grip something and thus starting the pulling of the at least one artificial tendon in order to enhance the gripping.

According to one embodiment at least one electrical wire is arranged at at least one patch. The electrical wiring may, for example, connect a sensor and an actuator for pulling of the at least one artificial tendon.

According to a second aspect of the invention a grip strengthening finger glove having a finger glove liner according to any of the above embodiments is provided.

### SHORT DESCRIPTION OF THE DRAWINGS

The present invention will now be described in more detailed under referral to the attached drawings, in which:

FIG. 1 shows an embodiment of a finger portion of a finger glove liner.

FIG. 2 shows an embodiment of a dorsal side of a finger glove liner.

FIG. 3 shows an embodiment of a ventral side of a finger glove liner.

FIG. 4 shows an embodiment of a grip strengthening finger glove.

### DETAILED DESCRIPTION OF EMBODIMENTS

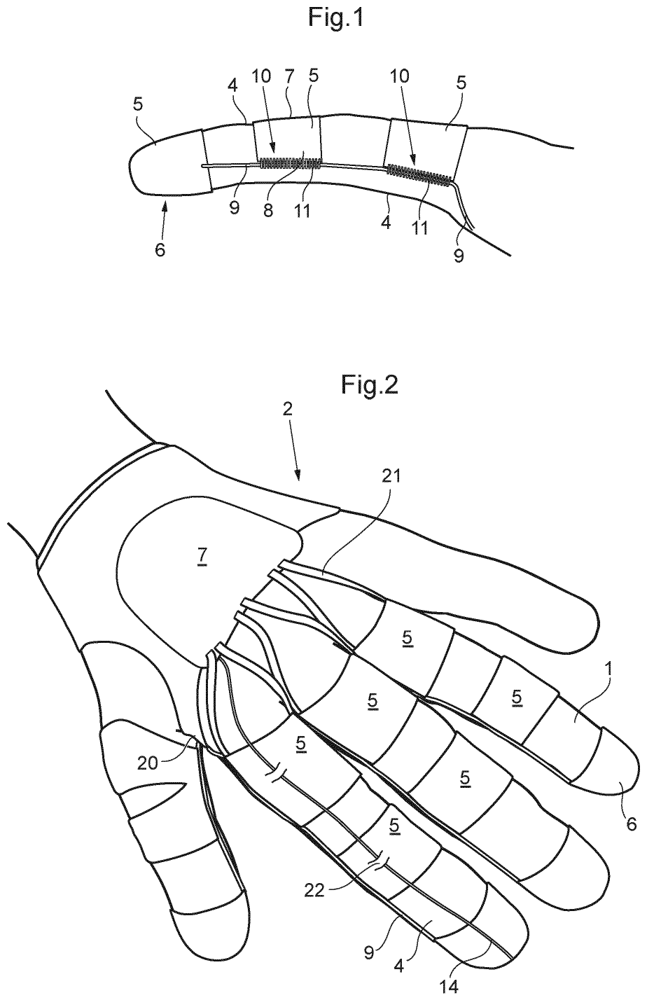

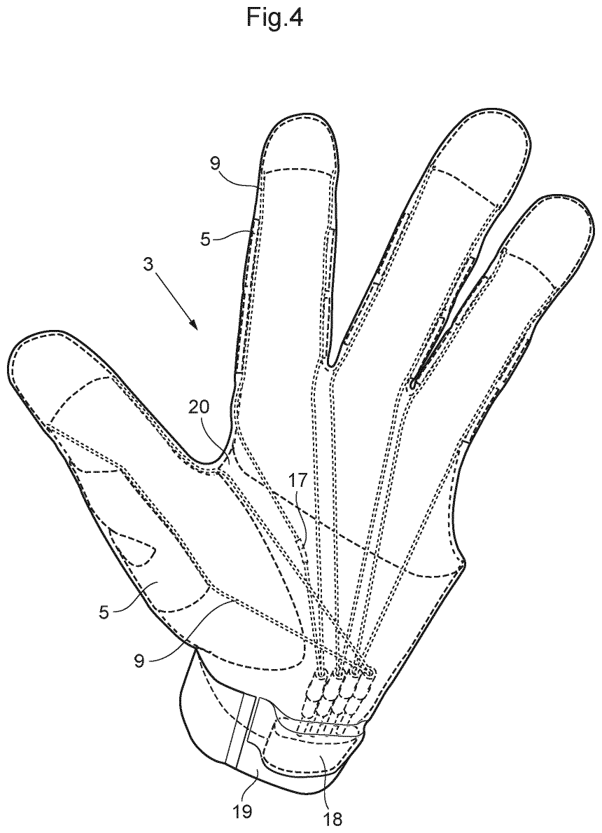

In FIG. 1 a finger portion 1 of a finger glove liner 2 is shown. The finger portion 1 may be suitable for any of the fingers of a wearer's hand including the thumb. A finger glove liner 2 comprises at least one finger portion 1. The finger glove liner 2 is supposed to be provided inside a finger glove 3 making up a grip strengthening finger glove, see FIG. 4.

The finger portion 1 and the finger glove liner 2 comprises a support material 4, which is stretchable. Thus one size of a finger glove liner 2 suits two or preferably three sizes of hands. For example, the material 4 may be a stitched material in order to provide the stretchability. Outside of the stretchable support material 4 at least one patch 5 is arranged. The patch 5 is preferably made of a material which is flexible and non-stretchable in a direction crosswise the length direction of the finger portion 1. In FIG. 1 a patch 5 over a tip portion 6 and two patches 5 along the length of the finger portion 1 are arranged. Preferably the patches 5 are arranged on a dorsal side 7 of the finger portion 1. It is preferred that the patches 5 reach down on at least one side 8 of the finger portion 1, preferably on both sides 8. Patches 5 may be provided at further parts of the finger glove liner 2, too, which will be described further below.

At least one artificial tendon 9 is attached at at least one patch 5. In the shown embodiment of FIG. 1 the artificial tendon 9 is slidably attached at the patch 5 at the tip portion 6 and along the side 8 portion of the patches 5 along the length of the finger portion 1. The artificial tendon 9 may be attached by means of, for example, loops (not shown) or a tunnel 10, such as a tunnel made up by stitches 11, which is shown in FIG. 1.

The patches 5 are arranged at the stretchable material 4 in such a way that when the stretchable material 4 stretches, the patches 5 will move to a proper position also for a larger hand than a hand fitting within the finger glove liner 2. This is possible when the patches 5 are only attached at the stretchable material 4 at points. In the non-stretched situation, the non-stretchable patches 5 are attached in bulky way such that the length of the patches 5 curves above the stretchable material 4. Thus, when the stretchable material 4 is stretched the patches 5 will straighten out and be flat along the surface of the stretchable material 4. When stretched even further, there will be spaces between patches.

It is also conceivable to have at least one patch 5 more or less encircling the finger portion 1 and attaching an artificial tendon 9 centrally at the ventral side of the finger portion 1 (not shown).

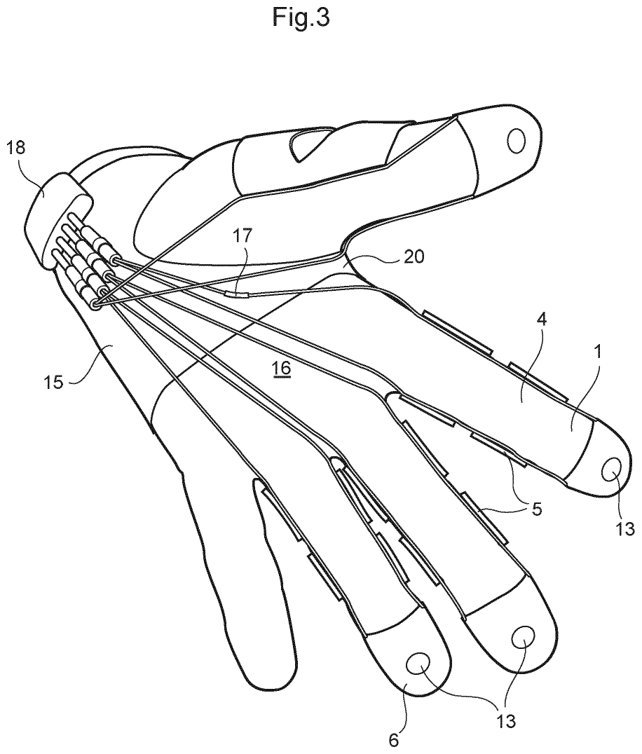

In FIGS. 2 and 3 an embodiment of a finger glove liner 2 is shown. Preferably at least one patch 5 is arranged on a ventral side 15 of the finger glove liner 2 at a hand portion 16. This ventral side 15 patch 5 has a guiding 17 for the at least one artificial tendon 9. The ventral side 15 patch 5 may be connected with a patch 5 arranged on the dorsal side 7 of the hand portion 16 of the finger glove liner 2. The connection 20 between the ventral side 15 patch 5 and the dorsal side 7 patch may be stretchable. Preferably a proximal patch 5 at the finger portion 1 is connected with means 21 at the dorsal side 7 patch 5 in order to inhibit sliding of the proximal patch 5 too long towards the distal end of the finger portion 1.

The at least one artificial tendon 9, in the shown embodiment two artificial tendons 9 run along each finger portion 1 possibly into a guiding 17, which guides the artificial tendons 9 towards an actuator 18 for example arranged at a wrist portion 19 of the grip strengthening finger glove 3, see FIG. 4.

The actuator 18 can pull the artificial tendons 9 when being given a signal. The signal will be achieved from at least one sensor 13 arranged in the finger glove liner 2. It is preferred that the at least one sensor 13 is attached at the tip portion 6, preferably at the patch 5 there. Obviously, sensors 13 may be arranged at more positions of the finger glove liner. At least one electrical wire 14 is attached, for example for connection of the at least one sensor 13 with a control means (not shown). In FIG. 2 a possible way of attaching the wire 14 is shown. Slits 22 are provided in the patches 5 and the wire 14 is threaded through the slits 22.

In the present application the embodiments in FIGS. 2-4 show a grip strengthening finger glove 3 for all fingers except the little finger, including the thumb. Although it is possible to provide a grip strengthening finger glove 3 having all fingers and strengthening arrangement for all fingers, or only one finger, or the thumb and the index finger, or the thumb and the two middle fingers, or in any other combination.

1. A finger glove liner comprising at least one finger portion, for arrangement inside an outer finger glove, making up a grip strengthening finger glove, the finger glove liner being made of a stretchable material in order to suit more than one size of hands, wherein at least one patch of material, which has attached thereto at least one artificial tendon, is arranged at the outside of the finger glove liner, wherein the at least one patch is arranged on a dorsal side of a finger portion of the finger glove liner, the at least one patch reaches over each of the sides of a finger portion of the finger glove liner with a side portion on each side and the at least one artificial tendon is arranged at a side portion.

2. The finger glove liner according to claim 1, wherein the at least one patch of material is made of a flexible and non-stretchable material in a direction crosswise the length direction of the finger portion 1.

3. The finger glove liner according to claim 1, wherein the patches are arranged freely from each other in order to be able to transfer with the expansion of the stretchable liner in use.

4. The finger glove liner according to claim 1, wherein the at least one patch distributes the force from the at least one artificial tendon attached to said patch.

5-6. (canceled)

7. The finger glove liner according to claim 1, wherein the at least one artificial tendon is arranged at a side portion, along the edge of the side portion of the patch.

8. The finger glove liner according to claim 1, wherein at least one sensor is arranged at the at least one patch.

9. The finger glove liner according to claim 1, wherein at least one electrical wire is arranged at the at least one patch.

10. A grip strengthening finger glove having a finger glove liner according to claim 1.
